(19) United States
(12) Patent Application Publication
(10) Pub. No.: US 2008/0283384 A1
(43) Pub. Date: Nov. 20, 2008

(54) Process for Producing Trioxane and at Least One Comonomer

(75) Inventors: Neven Lang, Mannheim (DE); Ralf Boehling, Lorsch (DE); Achim Stammer, Freinsheim (DE); Jan Oldenburg, Ludwigshafen (DE); Markus Siegert, Heidelberg (DE); Eckhard Stroefer, Mannheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/091,976

(22) PCT Filed: Oct. 27, 2006
(86) PCT No.: PCT/EP2006/067851

§ 371 Date: Apr. 29, 2008

(30) Foreign Application Priority Data
Oct. 31, 2005 (DE) 10 2005 051 974.1

(51) Int. Cl. C08G 2/10; B01D 3/34; C07D 317/12; C07D 323/06

(52) U.S. Cl. 203/34; 203/38

(57) ABSTRACT

The invention relates to a process for preparing trioxane and at least one comonomer for preparing (co)polymers based on trioxane, in which, in a first step, formaldehyde the at least one comonomer reactant are reacted in aqueous solution to give trioxane and comonomer, to obtain a reaction mixture A1 comprising trioxane, comonomer, formaldehyde and water, with or without comonomer reactant. In a second step, the reaction mixture A1 is distilled in a first distillation stage at a first pressure to obtain a stream B1 enriched in trioxane and comonomer and a stream B2 comprising essentially water and formaldehyde, with or without comonomer reactant. In a third step, stream B1 is distilled in a second distillation stage at a pressure which is above the pressure of the first distillation stage to obtain a stream C1 comprising trioxane, comonomer and water and a product stream C2 consisting essentially of comonomer and trioxane.

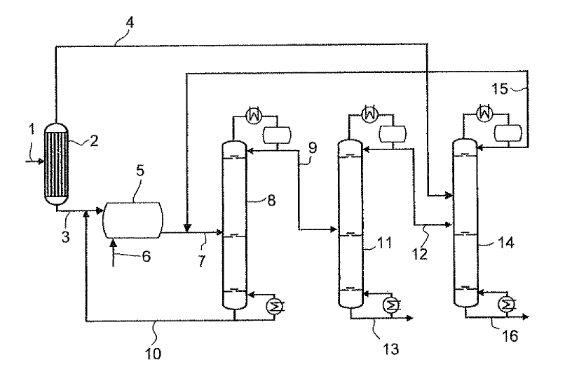

The present invention relates to a process for the combined preparation of trioxane and at least one further product (comonomer) formed by reaction of formaldehyde and a further reactant (comonomer reactant).

The trioxane is preferably used to prepare polyoxymethylene (POM). For stabilization, a comonomer is frequently polymerized into the POM. Suitable comonomers are, for example, dioxolane or butanediol formatl.

In the processes known from the prior art, the trioxane and the comonomer required to prepare POM are prepared in separate processes. For example, the preparation of 1,3,5-trioxane is known from DE-A 1 668 687. The 1,3,5-trioxane is prepared by distilling aqueous formaldehyde solutions in the presence of acidic catalysts. The trioxane is removed by extraction from the mixture which is formed in the reaction and comprises water, formaldehyde and trioxane.

DE-A 197 32 291 discloses a process for removing trioxane from the mixture comprising trioxane, formaldehyde and water, in which trioxane is first withdrawn from the mixture by pervaporation and the trioxane-enriched mixture is then separated by rectification into trioxane and a mixture comprising trioxane, formaldehyde and water.

A process for preparing dioxolane is described in DE-A 1 914 209. In this process, in the presence of a strongly acidic cation exchanger as a catalyst, ethylene glycol is reacted with aqueous formaldehyde to give dioxolane. The process is preferably carried out in such a way that the starting materials are used in approximately stoichiometric amounts, i.e. in a molar ratio of 1:1 of alcohol to formaldehyde. However, the process also works in principle satisfactorily at other quantitative ratios. The resulting, generally water-containing acetal is worked up, for example, by dewatering with solid alkali or concentrated alkali metal hydroxide solution, or by distillation.

A process for purifying dioxolane which has been prepared by reaction of ethylene glycol and formaldehyde in the presence of catalysts such as sulfuric acid, boron trifluoride, zinc chloride or acidic ion exchangers is known, for example, from DE-A 1 279 025. In this process, the vaporous, water-containing crude dioxolane is first fed to a column and distilled azeotropically, the exiting distillate having a maximum water content of 10% after it has been cooled in countercurrent with alkali metal hydroxide and/or a concentrated aqueous alkali metal hydroxide solution is treated and the treated product is finally fractionally distilled, the dioxolane being drawn off at the column bottom.

A further process for purifying dioxolane is known from DE-A 1 172 687. In this process, the crude dioxolane is treated with an inert organic liquid which is not miscible with it in every ratio and does not comprise any elements eliminable under the process conditions nor is capable of forming any compounds of such elements under the process conditions, in such a ratio that a layer separation occurs. The dioxolane-containing layer is removed and treated with aqueous alkali metal or alkaline earth metal hydroxide solution or with an alkali metal oxide or alkaline earth metal oxide or with an alkali metal or alkaline earth metal. After the dioxolane-containing liquid has been removed, it is distilled and the resulting, purified dioxolane is, if appropriate, subjected to an after treatment by filtration through a molecular sieve.

It is an object of the present invention to provide a process in which trioxane and a copolymer required for the preparation of POM are prepared in an energetically favorable manner.

The object is achieved by a process for preparing trioxane and at least one comonomer which is obtained by reacting formaldehyde with at least one comonomer reactant for preparing (co)polymers based on trioxane, which comprises the following steps:
a) reacting formaldehyde and the at least one comonomer reactant in aqueous solution to give trioxane and comonomer in a synthesis stage to obtain a reaction mixture A1 comprising trioxane, formaldehyde, water and comonomer, with or without unconverted comonomer reactant,
b) distilling reaction mixture A1 in a first distillation stage at a first pressure to obtain a stream B1 enriched in trioxane and comonomer and a stream B2 comprising substantially water and formaldehyde, with or without comonomer reactant,
c) distilling stream B1 in a second distillation stage at a pressure which is above the pressure of the first distillation stage to obtain a stream C1 comprising trioxane, comonomer and water and a product stream C2 comprising essentially comonomer and trioxane.

According to the invention, in a first step, an aqueous formaldehyde solution and at least one comonomer reactant are fed to a reactor. In the reactor, formaldehyde is firstly converted to trioxane, and the at least one comonomer reactant secondly reacts with formaldehyde to give the comonomer. The reaction is generally carried out at a pressure in the range from 0.5 to 10 bar, preferably in the range from 0.75 to 7 bar and in particular in the range from 0.8 to 4 bar, and a temperature in the range from 60 to 190° C., preferably in the range from 75 to 150° C. and in particular in the range from 80 to 130° C.

Comonomers which are prepared by the process according to the invention are, for example, cyclic ethers of the formula (I)

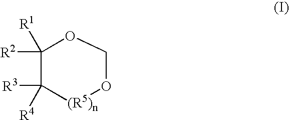

where R¹ to R⁴ are independently hydrogen, a C₁ to C₄-alkyl or halogen-substituted alkyl group having from 1 to 4 carbon atoms, and R⁵ is CH₂, CH₂O, a C₁ to C₄-alkylene or a C₁- to C₄-haloalkyl-substituted methylene group or a corresponding oxymethylene group, and n is an integer in the range from 0 to 3. Cyclic ethers suitable as comonomers are, for example, ethylene oxide, 1,2-propylene oxide, 1,2-butylene oxide, 1,3-butylene oxide, 1,3-dioxane, 1,3-dioxolane and 1,3-dioxepane which is also referred to as butanediol formal.

Likewise preparable as copolymers are bifunctional compounds of the formula (II)

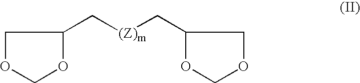

where Z is —O— or —ORO—, R is C₁ to C₈-alkylene or a C₃ to C₈-cycloalkylene, and m is 0 or 1. Preferred comonomers of this type are ethylene diglycide, diglycidyl ether and diethers of glycides and formaldehyde, dioxane or trioxane in a molar ratio of 2:1, and also diethers of 2 mol of glycidyl compounds and 1 mol of an aliphatic diol having from 2 to 8 carbon atoms, for example the diglycidyl ether of ethylene glycol, of 1,4-butanediol, of 1,3-butanediol, of cyclobutane-1,3-diol, of 1,2-propanediol and of cyclohexane-1,4-diol.

The at least one comonomer reactant is in each case selected such that reaction with formaldehyde under the conditions in the reactor generates the desired comonomer.

As the comonomer prepared in the process according to the invention, particular preference is given to 1,3-dioxolane. The comonomer reactant which is used to prepare the 1,3-dioxolane is ethylene glycol which reacts with the formaldehyde with elimination of water to give 1,3-dioxolane.

The reactions are generally carried out in the presence of an acidic catalyst. The pKa value of the catalyst is preferably less than 4. Suitable catalysts are, for example, organic or mineral acids, boron trifluoride, zinc chloride or acidic ion exchangers. The catalyst may be present in homogeneous or heterogeneous form.

A suitable reactor for carrying out the synthesis stage is any reactor known to those skilled in the art. However, preference is given to reactors in which the reaction can be carried out continuously. Such reactors are, for example, stirred tanks, delay tanks, tubular reactors, evaporators of various designs, column bottoms or else columns with suitable reaction zone. The selection of suitable columns is generally not critical in connection with the present invention. Suitable columns are known to those skilled in the art.

When a heterogeneous catalyst is used, it is present, for example, in the form of granule or in the form of packing. In this context, any packing known to those skilled in the art is conceivable. For example, structured packings, knitted fabrics, woven fabrics or random packings may be used. In this case, the catalyst is preferably present in the form of a coating on a support material. Suitable support materials are, for example, zeolites or phenol- or styrene-based resins. However, it is additionally also possible that the entire packing consists of the catalyst material.

After the reaction in step a), the reaction mixture thus obtained is distilled at a first pressure in a first distillation stage in step b). This pressure corresponds preferably to the pressure at which the formaldehyde and the at least one comonomer reactant have been converted to trioxane and comonomer. In this case, pressure differences can arise, for example, as a result of a pressure drop in the reactor or in pipelines which connect the reactor to the first distillation stage.

However, it is also possible to decompress the reaction mixture to a lower pressure or to compress it to a higher pressure before entry into the first distillation stage. However, the pressure of the first distillation stage preferably corresponds to the pressure of the reaction. The first distillation stage is generally operated at a pressure in the range from 0.2 to 10 bar, preferably in the range from 0.4 to 5 bar and in particular in the range from 0.5 to 2.5 bar.

In the first distillation stage, a stream B1 enriched in trioxane and comonomer and a stream B2 comprising substantially water and formaldehyde, with or without comonomer reactant, are obtained. The distillation may be carried out in any distillation apparatus known to those skilled in the art. Preference is given to a distillation column. Suitable distillation columns are, for example, packed columns or tray columns. Suitable packings are, for example, structured packings, woven fabrics, knitted fabrics or random packings. When a tray column is used, any trays known to those skilled in the art can be used.

The column of the first distillation stage comprises generally from 2 to 50 theoretical plates. The column of the first distillation stage preferably comprises from 4 to 25 theoretical plates.

The reaction mixture which is fed to the first distillation stage comprises generally from 0.1 to 25% by weight of trioxane, from 0.1 to 15% by weight of comonomer, from 20 to 80% by weight of formaldehyde, from 1 to 79.8% by weight of water and from 0 to 10% by weight of comonomer reactant. The reaction mixture preferably comprises from 0.4 to 20% by weight of trioxane, from 0.3 to 10% by weight of comonomer, from 30 to 69% by weight of formaldehyde, from 1 to 69% by weight of water and from 0 to 7% by weight of comonomer reactant.

The stream B1 enriched in trioxane and comonomer comprises generally from 25 to 80% by weight of trioxane, from 10 to 65% by weight of comonomer, from 1 to 20% by weight of formaldehyde and from 5 to 25% by weight of water. Stream B1 comprises preferably from 30 to 60% by weight of trioxane, from 15 to 60% by weight of comonomer, from 1 to 15% by weight of formaldehyde and from 5 to 20% by weight of water. Stream B2 comprises generally from 40 to 75% by weight of formaldehyde, from 15 to 50% by weight of water and from 5 to 50% by weight of the at least one comonomer reactant. Stream B2 comprises preferably from 40 to 75% by weight of formaldehyde, from 15 to 50% by weight of water and from 10 to 40% by weight of the at least one comonomer reactant. In addition, stream B2 may comprise not more than 5% by weight, preferably not more than 3% by weight and in particular not more than 2% by weight of trioxane and comonomer.

In a preferred embodiment, steps a) and b) are carried out together in one reactive distillation column. In this case, the reaction is generally effected in the lower part of the column. The reaction is preferably carried out under such conditions that the resulting reaction products are present in gaseous form. In exothermic reactions, it is also possible to utilize the heat of reaction formed in the reaction to evaporate the reaction products.

In the reactive distillation column, the separation into the lower-boiling stream B1 enriched in trioxane and comonomer and the high-boiling stream B2 comprising substantially water and formaldehyde, with or without comonomer reactant, is effected in a distillation part of the column which adjoins the reaction part.

When a reactive distillation column is used, the reactants are preferably added at the bottom of the column; the high-boiling stream B2 comprising substantially water and formaldehyde, with or without comonomer reactant, is preferably returned as liquid phase into the reaction part of the column; the stream B1 enriched in trioxane and comonomer is drawn off via the top of the reactive distillation column.

When the reaction in step a) and the first distillation stage b) are carried out in two different apparatuses, the reaction mixture A1 which is obtained in the reaction and comprises trioxane, comonomer, formaldehyde and water, with or without comonomer reactant, is added to the distillation column in which the first distillation stage b) is carried out preferably as a side feed in gaseous or liquid form. The stream B1 enriched in trioxane and comonomer is preferably withdrawn as a top draw stream and the stream B2 comprising substantially water and formaldehyde, with or without comonomer reactant, as a bottom draw stream.

The second distillation stage of step c) is generally carried out in a second distillation column. Suitable distillation columns for carrying out the second distillation stage are, for example, tray columns or packed columns. When a tray column is used, any and all trays known to those skilled in the art may be used. When a packed column is used, the packings used may be structured packings, woven fabrics, knitted fabrics or random packings.

The distillation of step c) is generally carried out at a pressure which is above the pressure of the first distillation stage. In general, the pressure of the second distillation stage is in the range between 0.2 and 17.5 bar, preferably in the range between 2 and 15 bar and more preferably in the range between 2.5 and 10 bar. The pressure of the second distillation stage is preferably at least 0.5 bar, more preferably at least 1 bar and in particular at least 3 bar higher than the pressure of the first distillation stage.

In the distillation of the second distillation stage, the stream B1 enriched in trioxane and comonomer is separated into a stream C1 comprising trioxane, comonomer, water and formaldehyde and a product stream C2 comprising substantially comonomer and trioxane. Stream C1 comprises generally from 15 to 60% by weight of trioxane, from 15 to 70% by weight of comonomer, from 10 to 30% by weight of water and from 1 to 20% by weight of formaldehyde, preferably from 10 to 55% by weight of trioxane, from 20 to 65% by weight of comonomer, from 15 to 25% by weight of water and from 2 to 15% by weight of formaldehyde. Stream C2 comprises generally from 0.1 to 7% by weight of comonomer and from 93 to 99.9% by weight of trioxane, preferably from 0.1 to 5% by weight of comonomer and from 95 to 99.9% by weight of trioxane Stream C2 may additionally comprise up to 2% by weight of water and formaldehyde.

Stream B1 is added to the second distillation column preferably as a side feed, and stream C1 is withdrawn as a top draw stream and stream C2 as a bottom draw stream.

In a preferred embodiment, the process additionally comprises the following steps:
d) distilling the stream C1 in a third distillation stage to obtain a stream D1 comprising trioxane, comonomer, formaldehyde and water and a stream D2 consisting substantially of water,
e) recycling stream D1 into the first distillation stage b).

The third distillation stage is preferably carried out in a third distillation column. The third distillation column is generally a packed column or tray column.

The distillation column of the third distillation stage has generally at least 2 theoretical plates, preferably from 5 to 50 theoretical plates and in particular from 10 to 25 theoretical plates.

The pressure of the third distillation stage c) is generally in the range from 0.2 to 25 bar, preferably in the range from 2 to 20 bar and in particular in the range from 2.5 to 15 bar. The pressure of the third distillation stage may be greater than, less than or equal to the pressure of the second distillation stage.

The stream D1 obtained in the distillation in the third distillation stage comprises generally from 15 to 70% by weight of trioxane, from 10 to 75% by weight of comonomer, from 5 to 20% by weight of formaldehyde and from 0 to 20% by weight of water, preferably from 20 to 60% by weight of trioxane, from 15 to 75% by weight of comonomer, from 5 to 15% by weight of formaldehyde and from 0 to 15% by weight of water.

In the context of the present invention, consisting substantially of water means that at least 90% by weight of water, preferably at least 93% by weight of water and in particular more than 95% by weight of water are present.

In order to prevent reactants or reaction products, each of which are products of value, from being discharged from the process as a waste stream, the stream D1 comprising the trioxane, comonomer, formaldehyde and water products of value is, in a preferred embodiment, recycled into the first distillation stage b). When this is done, a steady-state formaldehyde concentration is established. A portion of the formaldehyde present in stream D1 is removed in the first distillation column and fed back to the reactor in stream B2.

In a further embodiment, the process additionally comprises the following step:
f) concentrating an aqueous formaldehyde solution E1 in a formaldehyde concentration unit which is connected upstream of the synthesis stage to obtain a low-formaldehyde stream E2 and a formaldehyde-rich stream E3, and feeding the formaldehyde-rich stream E3 to the synthesis stage.

The aqueous formaldehyde solution E1 fed to the concentration unit comprises generally from 25 to 65% by weight of formaldehyde and from 35 to 75% by weight of water, preferably from 30 to 60% by weight of formaldehyde and from 40 to 70% by weight of water. The formaldehyde-rich stream E3 obtained in the concentration comprises generally at least 50% by weight of formaldehyde, preferably at least 55% by weight of formaldehyde. The low-formaldehyde stream E2 comprises generally at most 35% by weight of formaldehyde, preferably at most 30% by weight of formaldehyde.

Suitable concentration units are, for example, evaporators or distillation columns. All evaporator designs known to those skilled in the art are suitable. Preference is given to continuous evaporators, for example forced-circulation evaporators, falling-film evaporators, thin-layer evaporators, helical-tube evaporators or any other continuous evaporators known to those skilled in the art. Particularly preferred evaporators are falling-film evaporators.

When a distillation column is used as the formaldehyde concentration unit, any distillation column known to those skilled in the art can be used. Suitable distillation columns are, for example, tray columns or packed columns. Suitable packings are, for example, structured packings, woven fabrics, knitted fabrics or random packings.

The concentration of the aqueous formaldehyde solution is carried out generally at a pressure in the range from 0.05 to 1 bar and a temperature in the range from 40 to 98° C.

The formaldehyde-rich stream E3 obtained in the concentration is obtained preferably as a bottom draw stream and the low-formaldehyde stream E2 as a top or vapor draw stream. The low-formaldehyde stream E2 is preferably fed to the third distillation stage.

In addition to water, formaldehyde, trioxane, comonomer and comonomer reactant which may be present, up to 15% by weight, generally from 1 to 10% by weight of low boilers may be present especially in streams A1 and B1. Typical low boilers which can be formed in the synthesis and the subsequent distillative separation are methyl formate, methylal, dimethoxydimethyl ether, trimethoxydimethyl ether, methanol, formic acid, and also further hemiacetals and full acetals, and secondary components caused by the particular comonomer reactant.

The low boilers which may be present in streams A1 and B1 may, in a further embodiment, be removed in a low boiler removal stage. For this purpose, the process additionally comprises the following step:
g) distilling stream B1 in a low boiler removal stage at a pressure between 1 and 3 bar to obtain a stream B1″ comprising low boilers and a stream B1′ comprising trioxane, comonomer, formaldehyde and water, and feeding stream B1′ as stream B1 to the second distillation stage c).

The low boiler removal stage is generally likewise carried out in any distillation column. Suitable distillation columns here too are both tray columns and packed columns.

When the low boiler removal stage is carried out in a fourth distillation column, stream B1 is preferably fed as a side feed, and stream B1″ is preferably withdrawn as a top draw stream and stream B1′ preferably as a bottom draw stream.

The distillation column of the low boiler removal stage comprises generally at least 2 theoretical plates, preferably from 4 to 50 theoretical plates and in particular from 4 to 40 theoretical plates.

The distillation of the low boiler removal stage is preferably carried out at a pressure in the range from 1 to 2.5 bar and a temperature in the range from 60 to 140° C.

The invention will be described in detail hereinafter with reference to a drawing.

The single FIGURE shows a process flow diagram of the process according to the invention for preparing trioxane and comonomer.

An aqueous formaldehyde solution 1 (stream E1) is fed to a concentration unit 2. An example of a suitable concentration unit is an evaporator or a distillation column. In the concentration unit 2, the aqueous formaldehyde solution is separated into a formaldehyde-rich stream 3 (stream E3) and a low-formaldehyde stream 4 (stream E2). The formaldehyde-rich stream 3 is fed to a reactor 5. In addition to the formaldehyde-rich stream 3, at least one comonomer reactant 6 which reacts by reaction with formaldehyde to give a comonomer which is used to prepare (co)polymers based on trioxane is fed to the reactor. The comonomer reactant 6 may either be fed directly to the reactor or be mixed with the formaldehyde-rich stream 3 before the addition into the reactor 5 and fed to the reactor 5 together with it. In the reactor 5, the formaldehyde and the comonomer reactant are converted in aqueous solution to give trioxane and comonomer, to obtain a reaction mixture 7 (stream A1) comprising trioxane, comonomer, formaldehyde and water, with or without comonomer reactant.

The reaction mixture 7 is fed to a first distillation column 8. The addition is effected preferably via a side feed. In the first distillation column 8, the reaction mixture is distilled into a stream 9 (stream B1) enriched in trioxane and comonomer and a stream 10 (stream B2) comprising substantially water and formaldehyde, with or without comonomer reactant. The stream 9 enriched in trioxane and comonomer is withdrawn from the first distillation column 8 via the top and the stream 10 comprising substantially water and formaldehyde, with or without comonomer reactant, at the bottom. The pressure at which the first distillation column 8 is operated corresponds preferably to the pressure in the reactor 5. In order to be able to achieve higher formaldehyde concentrations, it is, however, also possible to operate the reactor at a higher pressure than the first distillation column.

The stream 10 comprising essentially water and formaldehyde, with or without comonomer reactant, is recycled into the reactor 5. The stream 10 may either be added directly to the reactor 5 or be mixed with the formaldehyde-rich stream 3 before the addition into the reactor 5 and then added to the reactor 5 together with it.

In addition to the embodiment shown in the FIGURE, in which the reactor 5 and the first distillation column 8 are two separate apparatuses, it is also possible to use one reactive distillation column, in which case the reaction of the formaldehyde and of the at least one comonomer reactant to give trioxane and comonomer is effected in the bottom of the column and the distillative separation is carried out in the column attached directly thereto.

The stream 9 enriched in trioxane and comonomer is fed to a second distillation column 11. It is preferably fed as a side feed. In the second distillation column 11, the stream 9 enriched in trioxane and comonomer is distilled into a stream 12 (stream C1) comprising trioxane, comonomer and water and a product stream 13 (stream C2) comprising substantially comonomer and trioxane. The stream 12 comprising trioxane, comonomer and water is withdrawn from the second distillation column via the top and the product stream 13 at the bottom. The distillation in the second distillation column 11 is carried out at a pressure which is higher than the pressure at which the first distillation column 8 is operated.

The stream 12 comprising trioxane, comonomer and water is fed to a third distillation column 14. The stream 12 comprising trioxane, comonomer and water is added preferably as a side feed. In addition, the low-formaldehyde stream 4 which is obtained in the concentration unit 2 is fed to the third distillation column. The streams 4, 12 can be added as two separate feeds, preferably two side feeds or as one common feed. In the case of common addition, the streams 4, 12 are mixed before the addition. In the third distillation column, the distillation affords a stream 15 (stream D1) comprising trioxane, comonomer, formaldehyde and water and a stream 16 (stream D2) consisting substantially of water. The stream 15 comprising trioxane, comonomer, formaldehyde and water is withdrawn via the top and the stream 16 consisting essentially of water at the bottom of the third distillation column 14.

The stream 15 comprising trioxane, comonomer, formaldehyde and water is recycled into the first distillation column 8. The addition can be effected either directly as a side feed into the first distillation column 8 or together with the reaction mixture 7, in which case the reaction mixture 7 and the stream 15 comprising trioxane, comonomer, formaldehyde and water are mixed before addition into the first distillation column 8.

### EXAMPLES

### Comparative Example

6 kg/h of an aqueous formaldehyde solution composed of 50% by weight of water and 50% by weight of formaldehyde are fed to a falling-film evaporator as a concentration unit. In the concentration unit, this is concentrated to give a formaldehyde-rich stream of 4.4 kg/h with a composition of 60% by weight of formaldehyde and 40% by weight of water. The formaldehyde-rich stream is fed to a reactive distillation column together with a top draw stream of a third distillation column comprising 70.9% by weight of trioxane, 18.0% by weight of water and 11.1% by weight of formaldehyde. The mass flow rate of the top draw stream of the third distillation column is 11.1 kg/h. In the reactive distillation column, the formaldehyde is converted to trioxane in an equilibrium reaction at a temperature of 115° C. and a pressure of 1.7 bar. The mixture formed is drawn off via the top of the reactive distillation column and is composed of 70% by weight of trioxane, 24% by weight of water and 6% by weight of formaldehyde. The mass flow rate of the stream drawn off at the top of the reactive distillation column is 15.5 kg/h. This stream is fed to a second distillation column and distilled therein at a bottom temperature of 178° C. and a pressure of 5.5 bar into a stream, drawn off at the top of the second distillation column, of 12.5 kg/h which comprises 62.9% by weight of trioxane, 29.7% by weight of water and 7.3% by weight of formaldehyde, and a product stream, drawn off at the bottom, of 3 kg/h which comprises 99.5% by weight of trioxane, 0.1% by weight of water and 0.4% by weight of formaldehyde. The stream drawn off at the top of the second distillation column is fed to the third distillation column together with the stream, obtained at the top of the concentration unit, of 1.6 kg/h with a composition of 20% by weight of formaldehyde and 80% by weight of water. In the third distillation column, the top draw stream fed to the reactive distillation column and a bottom draw stream of 3 kg/h composed of 99.9% by weight of water and 0.1% by weight of formaldehyde are obtained. The distillation in the third distillation column is carried out at a bottom temperature of 155° C. and a pressure of 5.5 bar.

### Example 1

6.8 kg/h of an aqueous formaldehyde solution composed of 50% by weight of water and 50% by weight of formaldehyde are fed to a falling-film evaporator as a concentration unit. In the concentration unit, this is concentrated to a formaldehyde-rich stream of 5.2 kg/h. Ethylene glycol is admixed to the formaldehyde-rich stream so that it has a composition of 59.9% by weight of formaldehyde, 40% by weight of water and 0.1%, by weight of ethylene glycol. The formaldehyde-rich stream is fed to a reactive distillation column together with a top draw stream of a third distillation column comprising 54.4% by weight of trioxane, 11.7% by weight of water, 25.3% by weight of dioxolane and 8.6% by weight of formaldehyde. The mass flow rate of the top draw stream of the third distillation column is 13.8 kg/h. In the reactive distillation column, the formaldehyde is converted to trioxane in an equilibrium reaction and ethylene glycol is reacted with formaldehyde to give dioxolane at a temperature of 113° C. and a pressure of 1.7 bar in the presence of sulfuric acid as a catalyst. The resulting mixture is drawn off via the top of the reactive distillation column and is composed of 57.3% by weight of trioxane, 19.6% by weight of water, 18.4% by weight of dioxolane and 4.7% by weight of formaldehyde. The mass flow rate of the stream drawn off at the top of the reactive distillation column is 19 kg/h. This stream is fed to a second distillation column and distilled therein at a bottom temperature of 167° C. and a pressure of 5 bar to a stream, drawn off at the top of the second distillation column, of 15.6 kg/h which comprises 48.1% by weight of trioxane, 23.9% by weight of water, 22.4% by weight of dioxolane and 5.6% by weight of formaldehyde, and a product stream, drawn off at the bottom, of 3.4 kg/h which comprises 99.4% by weight of trioxane, 0.1% by weight of water, 0.1% by weight of dioxolane and 0.4% by weight of formaldehyde. The stream drawn off at the top of the second distillation column is fed to the third distillation column together with the stream, obtained at the top of the concentration unit, of 1.6 kg/h having a composition of 20% by weight of formaldehyde and 80% by weight of water. In the third distillation column, the top draw stream fed to the reactive distillation column and a bottom draw stream of 3.4 kg/h composed of 99.9% by weight of water and 0.1% by weight of formaldehyde are obtained. The distillation in the third distillation column is carried out at a bottom temperature of 155° C. and a pressure of 5 bar.

### Example 2

11.6 kg/h of an aqueous formaldehyde solution composed of 50% by weight of water and 50% by weight of formaldehyde are fed to a falling-film evaporator as a concentration unit. In the concentration unit, this is concentrated to a formaldehyde-rich stream of 8.8 kg/h. Ethylene glycol is admixed to the formaldehyde-rich stream so that it has a composition of 59.6% by weight of formaldehyde, 39.7% by weight of water and 0.7% by weight of ethylene glycol. The formaldehyde-rich stream is fed to a reactive distillation column together with a top draw stream of a third distillation column comprising 22.7% by weight of trioxane, 0.3% by weight of water, 70.9% by weight of dioxolane and 6.1% by weight of formaldehyde. The mass flow rate of the top draw stream of the third distillation column is 22.5 kg/h. In the reactive distillation column, the formaldehyde is converted to trioxane in an equilibrium reaction and ethylene glycol is reacted with formaldehyde to give dioxolane at a temperature of 110° C. and a pressure of 1.7 bar in the presence of sulfuric acid as a catalyst. The resulting mixture is drawn off via the top of the reactive distillation column and is composed of 34.6% by weight of trioxane, 11.8% by weight of water, 50.9% by weight of dioxolane and 2.7% by weight of formaldehyde. The mass flow rate of the stream drawn off at the top of the reactive distillation column is 31.5 kg/h. This stream is fed to a second distillation column and distilled therein at a bottom temperature of 165° C. and a pressure of 5 bar to a stream, drawn off at the top of the second distillation column, of 25.6 kg/h which comprises 20.0% by weight of trioxane, 14.3% by weight of water, 62.4% by weight of dioxolane and 3.3% by weight of formaldehyde, and a product stream, drawn off at the bottom, of 5.9 kg/h which comprises 98.0% by weight of trioxane, 0.8% by weight of water, 1.0% by weight of dioxolane and 0.2% by weight of formaldehyde. The stream drawn off at the top of the second distillation column is fed to the third distillation column together with the stream, obtained at the top of the concentration unit, of 2.7 kg/h having a composition of 20% by weight of formaldehyde and 80% by weight of water. In the third distillation column, the top draw stream fed to the reactive distillation column and a bottom draw stream of 5.8 kg/h composed of 99.9% by weight of water and 0.1% by weight of formaldehyde are obtained. The distillation in the third distillation column is carried out at a bottom temperature of 155° C. and a pressure of 5 bar.

1-15. (canceled)

16. A process for preparing trioxane and at least one comonomer which is obtained by reacting formaldehyde with at least one comonomer reactant for preparing (co)polymers based on trioxane, comprising:
a) reacting formaldehyde and the at least one comonomer reactant in aqueous solution to give trioxane and comonomer in a synthesis stage to obtain a reaction mixture A1 comprising trioxane, comonomer, formaldehyde, and water, and optionally comonomer reactant;
b) distilling reaction mixture A1 in a first distillation stage at a first pressure to obtain a stream B1 enriched in trioxane and comonomer and a stream B2 comprising essentially water and formaldehyde, and optionally comonomer reactant;
c) distilling stream B1 in a second distillation stage at a pressure which is above the pressure of the first distillation stage to obtain a stream C1 comprising trioxane, comonomer and water and a product stream C2 consisting essentially of comonomer and trioxane.

17. The process of claim 16, wherein a) and b) are carried out at a pressure in the range of from 0.2 to 10 bar and c) is carried out at a pressure in the range of from 0.2 to 17.5 bar.

18. The process of claim 16, wherein a) is carried out in the presence of an acidic catalyst, wherein said acidic catalyst is optionally in heterogeneous or homogeneous form.

19. The process of claim 16, wherein a) and b) are carried out together in one reactive distillation column comprising a reaction part, wherein stream B2 is fed to said reaction part as liquid reflux.

20. The process of claim 16, wherein b) is carried out in a first distillation column to which reaction mixture A1 is added as a side feed and from which stream B1 is withdrawn as a top draw stream and stream B2 as a bottom draw stream, and c) is carried out in a second distillation column to which stream B1 is added as a side feed and from which stream C1 is withdrawn as a top draw stream and stream C2 as a bottom draw stream.

21. The process of claim 16, further comprising:
d) distilling stream C1 in a third distillation stage at a pressure above the pressure of the second distillation stage of c) to obtain a stream D1 comprising trioxane, comonomer, formaldehyde, and water and a stream D2 comprising water,
e) recycling stream D1 into the first distillation stage of b).

22. The process of claim 21, wherein d) is carried out at a pressure in the range of from 1 to 25 bar.

23. The process of claim 21, wherein d) is carried out in a third distillation column to which stream C1 is fed as a side feed and from which stream D1 is withdrawn as a top draw stream and stream D2 as a bottom draw stream.

24. The process of claim 21, further comprising:
f) concentrating an aqueous formaldehyde solution E1 in a formaldehyde concentration unit which is connected upstream of the synthesis stage to obtain a low-formaldehyde stream E2 and a formaldehyde-rich stream E3, and feeding said formaldehyde-rich stream E3 to the synthesis stage of a).

25. The process of claim 24, wherein said low-formaldehyde stream E2 is fed to the third distillation stage of d).

26. The process of claim 24, wherein the formaldehyde concentration unit is an evaporator or a distillation column, the formaldehyde-rich stream E3 is obtained as a bottom draw stream, and the low-formaldehyde stream E2 is obtained as a top or vapor draw stream.

27. The process of claim 26, wherein the evaporator is a falling-film evaporator.

28. The process of claim 24, which additionally comprises the following step:
g) distilling stream B1 in a low boiler removal stage at a pressure between 1 and 3 bar to obtain a stream B1″ comprising low boilers and a stream B1′ comprising trioxane, comonomer, formaldehyde and water, and feeding stream B1′ as stream B1 to the second distillation stage of c).

29. The process of claim 28, wherein g) is carried out in a fourth distillation column to which stream B1 is fed as a side feed and from which stream B1″ is withdrawn as a top draw stream and stream B1′ as a bottom draw stream.

30. The process of claim 16, wherein the comonomer reactant is ethylene glycol and the comonomer is dioxolane.

31. The process of claim 16, further comprising:
f) concentrating an aqueous formaldehyde solution E1 in a formaldehyde concentration unit which is connected upstream of the synthesis stage to obtain a low-formaldehyde stream E2 and a formaldehyde-rich stream E3, and feeding the formaldehyde-rich stream E3 to the synthesis stage of a).

32. The process of claim 30, further comprising:
g) distilling stream B1 in a low boiler removal stage at a pressure between 1 and 3 bar to obtain a stream B1″ comprising low boilers and a stream B1′ comprising trioxane, comonomer, formaldehyde and water, and feeding stream B1′ as stream B1 to the second distillation stage c).
